(19) United States
(12) Patent Application Publication
(10) Pub. No.: US 2007/0264250 A1
(43) Pub. Date: Nov. 15, 2007

(54) Composition for Skin-Whitening and Wrinkle-Care

(76) Inventor: Dong II Jang, Gyeonggi-do (KR)

Correspondence Address: GRAYBEAL, JACKSON, HALEY LLP, 155 - 108TH AVENUE NE, SUITE 350, BELLEVUE, WA, 98004-5973, US

(21) Appl. No.: 11/658,136

(22) PCT Filed: Jun. 10, 2004
(86) PCT No.: PCT/KR04/01388

§ 371 Date: Jan. 19, 2007

(51) Int. Cl. A61K 38/46; A61Q 19/02; A61Q 19/08

(52) U.S. Cl. 424/94.6

(57) ABSTRACT

The present invention relates to an external composition for skin, comprising 2-o-a-glucopyranosyl-L-ascorbic acid and a-glucosidase, which hydrolyzes 2-o-a-glucopyranosyl-L-ascorbic acid into ascorbic acid and glucose. In the composition of the present invention, 2-o-a-glucopyranosyl-L-asocorbic acid and a-glucosidase are contained in a formula, or separated in different formulas, which are mixed just before applying to the skin.

### BRIEF DESCRIPTION OF DRAWINGS

FIG. 1 is a graph showing stability of 2-o-α-glucopyranosyl-L-ascorbic acid and decomposition thereof by glucosidase in a composition according to the present invention.

### DETAILED DESCRIPTION OF INVENTION

### Object of Invention

### Technical Field of Invention and Related Prior Art

The present invention relates to an external preparation for skin, which assures stability of vitamin C.

It is clinically known that ascorbic acid, which is commonly called vitamin C, suppresses generation of melanin of pigment cells in epidermal tissue of the skin, and has a whitening function to reduce a concentration of melanin existing in the epidermis. It also increases generation of collagen in fibroblasts of a corium tissue to make the skin resilient and reduce wrinkles, thus being extensively used for cosmetics or skin care products.

However, since vitamin C is disadvantageous in that it is stable in a dried powder state but easily oxidized and degraded in an aqueous solution state, it is difficult to formulate it to produce cosmetics. Furthermore, it is more rapidly oxidized and degraded when it is in a neutral or alkaline pH condition, or when it is exposed to metal ions, such as iron or copper. Since the degradation of vitamin C reduces its titer and causes discoloration with time, it has a fatal disadvantage when used for medical products, and particularly, cosmetics.

With respect to stabilization of vitamin C, generally, many studies have been made in two directions. The first method is to prevent a vessel containing vitamin C from coming into contact with oxygen by charging of nitrogen, or from coming into contact with water by formation of an anhydrous formulation having no water or by capsulation, thereby preventing oxidation thereof. The second method is to force a hydroxyl group (OH) of C₂ to be derivatized through esterification bonding based on the fact that instability of vitamin C is caused by an enediol ring structure weak to oxidation.

Widely known examples of derivatives of vitamin C, which are used for stabilization, include L-ascorbic acid 2-o-phosphate, L-ascorbic acid 2-o-sulfate, 2-o-octadecyl ascorbic acid (CV-3611), and ascorbic acid 2-methylester (AA-2M). Stabilities of such derivatives are significantly improved in comparison with vitamin C. However, the stabilized derivatives which are bonded to phosphates or sulfates having an electric charge at secondary positions thereof are disadvantageous in that they are difficult to permeate through the skin, their price is a few tens of times as high as that of pure vitamin C, and solubility is insufficiently high. As well, the derivative which is bonded to an alkyl group at a secondary position thereof is problematic in that, even though it permeates well through the skin, activity of an enzyme for converting it into vitamin C is low in an epidermal layer, thus it is inefficient in terms of physiological activity.

### Technical Problem

Accordingly, an object of the present invention is to provide a novel vitamin C external preparation. The external preparation maintains high whitening, wrinkles reduction, and skin resilience improvement functions of vitamin C, seldom irritates the skin, easily permeates through the skin, and avoids instability of vitamin C.

In other words, the object of the present invention is to provide the novel vitamin C external preparation, which is stable and has excellent whitening function.

Another object of the present invention is to provide a novel vitamin C external preparation, which is stable and has excellent wrinkles reduction and skin resilience improvement functions.

### Construction of Invention

In order to accomplish the above objects, the present invention provides an external preparation for skin, which comprises 2-o-α-glucopyranosyl-L-ascorbic acid expressed by the following Formula (referred to as “AA-2G”) and α-glucosidase spatially separated from each other as effective components.

Structure of AA-2G

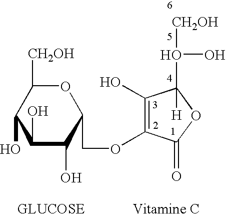

The present invention employs 2-o-α-glucopyranosyl-L-ascorbic acid and α-glucosidase which have excellent stability as derivatives of vitamin C. A composition of the present invention exists in a form of stable 2-o-α-glucopyranosyl-L-ascorbic acid before it is applied to the skin, and is converted into vitamin C after it is applied to the skin, thereby being easily absorbed by the skin and being safely applied to the skin because irritation of the skin is reduced due to low acidity of a vitamin C aqueous solution. 2-o-α-glucopyranosyl-L-ascorbic acid which is used in the present invention is a stabilized derivative of vitamin C, and is known as a natural compound which exists in tissues of mammals, such as rats and marmots, and particularly, in the small intestine and kidneys of living bodies. Unlike other derivatives, it is produced using an enzyme conversion technology instead of synthesis. Hence, it can be produced so as to have few impurities to irritate the skin.

2-o-α-glucopyranosyl-L-ascorbic acid is a compound in which a hydroxyl group is alpha-substituted by one glucose molecule at a secondary position of ascorbic acid and which has resistance to oxidation and excellent molecular stability in terms of physicochemical properties, unlike other conventional derivatives of vitamin C. 2-o-α-glucopyranosyl-L-ascorbic acid is slowly converted into vitamin C after it is applied to the skin in a form of external preparation for the skin according to the present invention, and then absorbed by the skin. In some cases when it is directly absorbed by skin tissue while it is not converted into vitamin C, ascorbic acid is cleaved by α-glucosidase existing in a living body, thus activity of vitamin C is assured.

As described below in Example 1, if 2-o-α-glucopyranosyl-L-ascorbic acid exists in a mixture form with glucosidase, it is readily decomposed into glucose and vitamin C, thus being easily degraded in an aqueous solution like typical vitamin C. Accordingly, in order to accomplish the present invention, 2-o-α-glucopyranosyl-L-ascorbic acid and glucosidase must be produced, delivered, and stored while they are spatially separated from each other. Various typical methods can be used to spatially separate two components. In detail, 2-o-α-glucopyranosyl-L-ascorbic acid and glucosidase may be separately contained in one vessel having an internal partition, or one of the two components may be produced in a form of capsule, microcapsule, or dispersant packed in particulate or lipid and then mixed with a solution containing the other component. Alternatively, they may be separately contained in different vessels. In this case, two components are mixed immediately before they are applied to the skin.

In the external composition for the skin according to the present invention, it is preferable that a content of 2-o-α-glucopyranosyl-L-ascorbic acid be 0.05-15.0 wt %, and preferably, 0.1-5.0 wt % based on the composition. It is preferable that glucosidase be added in 25-100 units based on 1 wt % of 2-o-α-glucopyranosyl-L-ascorbic acid. If the content of glucosidase is too low, since a conversion rate of 2-o-α-glucopyranosyl-L-ascorbic acid into vitamin C is low, permeation through the skin is poor, thus skin care performance of vitamin C is inferior to a level required in the present invention. Needless to say, it is unnecessary to add glucosidase in an amount that is more than the amount needed to achieve the conversion into vitamin C.

In the present invention, 2-o-α-glucopyranosyl-L-ascorbic acid is obtained from natural sources or biochemically synthesized. In nature, there exists ascorbic acid in a form of glucoside in plants or mushrooms. Accordingly, it is separated from natural extracts, or used in the form of unprocessed natural extracts in the composition of the present invention.

Furthermore, in the present invention, glucosidase may be added in a form of enzyme derived from a plant, such as rice, barley, oat, or almond, or a microorganism, such as Aspergillus, Saccharomyces, or Zymomonas, or in a form of enzyme which is genetically mass-produced by introducing glucosidase genes which are obtained from them into Escherichia coli. The enzyme may be purified, or be in a form of coarse extracts. In other words, in the present invention, glucosidase may be produced in a living body using gene recombination or separated from a natural source, and the enzyme source is not limited. As well, glucosidase may be added in a form of extract obtained from a plant or a microorganism containing the above-mentioned enzyme, and preferably a plant, such as almond, barley, or oat, or a microorganism, such as Aspergillus niger or Saccharomyces cerevisiae. Needless to say, the source of glucosidase is not limited to the above example. It is apparent to those skilled in the art that any glucosidase may be applied to the external composition for the skin according to the present invention regardless of its source as long as it hydrolyzes 2-o-α-glucopyranosyl-L-ascorbic acid to produce ascorbic acid and glucose.

2-o-α-glucopyranosyl-L-ascorbic acid and glucosidase of the composition according to the present invention are provided in different formulations, or two components are stored in a special vessel which has a diaphragm dividing an internal space and mixed immediately before they are applied to the skin. If 2-o-α-glucopyranosyl-L-ascorbic acid and glucosidase are mixed with each other, 2-o-α-glucopyranosyl-L-ascorbic acid is decomposed into ascorbic acid and glucose by glucosidase. Since ascorbic acid is rapidly absorbed on the skin (that is to say, a time which is required to absorb ascorbic acid after the decomposition into ascorbic acid is conducted is too short to oxidize ascorbic acid), it is not oxidized but maintained in an active form, thus whitening and wrinkles reduction functions are assured. Compared to a conventional product containing only ascorbic acid, which is readily deactivated and degraded during a storage period before application to the skin and thus does not maintain the desirable functions of ascorbic acid, the composition of the present invention has higher stability so as to assure intrinsic functions of vitamin C, resulting in excellent skin care performance.

The external composition for the skin according to the present invention may be added to various types of goods, such as a skin softener (skin tonic), a nourishing lotion (moisturizing lotion), a nourishing cream, a massage cream, an essence, or a pack. When the external composition for the skin according to the present invention is applied to cosmetics, if necessary, a predetermined amount of subsidiary additive, such as a whitening agent, a moisturizing agent, an antioxidant, a UV absorbing agent, a surfactant, a thickening agent, alcohol, a preservative, a gelling agent, perfume, a filler, or a dye, which is typically used as components constituting the cosmetics, may be added thereto. Preferably, the subsidiary additive is added in an amount which is typically used in a cosmetic field, for example, 0.01-20% based on the total weight of the composition.

The external composition for the skin according to the present invention may be a pharmaceutical formulation, such as a solution, a gel, a lotion, an emulsified dispersant, emulsion, a microcapsule, or a particulate-packed dispersant, which is typically used for local application. Particularly, if 2-o-α-glucopyranosyl-L-ascorbic acid and glucosidase are contained in the same formulation, one component of 2-o-α-glucopyranosyl-L-ascorbic acid and glucosidase may be produced in a form of a microcapsule, or a particulate- or lipid-packed dispersant using a technology typically used in the cosmetic field, so as to suppress mixing thereof until they are applied to the skin, resulting in maximized stability.

In an Example of the present invention, a quantitative analysis was conducted with an HPLC frequently used in a component analysis for organic material, resulting in the confirmation that 2-o-α-glucopyranosyl-L-ascorbic acid is completely decomposed by glucosidase into vitamin C and glucose.

Meanwhile, in another Example of the present invention, healthy adults of both sexes underwent a closure patch test, and primary irritation caused by vitamin C generated by glucosidase on the human body was evaluated. Generally, a concentration of vitamin C applied to the skin, which is clinically considered to be useful for wrinkle and whitening purposes, is 5% or more, thus it is applied in a high concentration formulation. Since this concentration of vitamin C is strongly acidic, it irritates the skin if it is directly applied to the skin, thus causing difficulty in development of an external preparation for skin using vitamin C. Unlike in a control group where vitamin C was directly patched, vitamin C was absorbed by the skin while it was cleaved by an enzyme reaction in a test group where a mixed solution of 2-o-α-glucopyranosyl-L-ascorbic acid and glucosidase was patched, thus primary irritation was largely prevented.

Furthermore, after UV was radiated onto the skin of healthy adults of both sexes to form a pigment in the skin, a preparation containing both 2-o-α-glucopyranosyl-L-ascorbic acid and glucosidase (they are separated from each other by a diaphragm in preparation, and mixed when discharged from the preparation) was applied thereto to evaluate whitening due to 2-o-α-glucopyranosyl-L-ascorbic acid and glucosidase. The external composition for the skin according to the present invention has superior whitening ability in comparison with use of only 2-o-α-glucopyranosyl-L-ascorbic acid or only vitamin C.

With respect to conventional technologies related to an external preparation for skin containing vitamin C, a prior patent (Korean Patent Laid-Open Publication No. 2003-0032195) discloses direct use of 2-o-α-glucopyranosyl-L-ascorbic acid. However, it is different from the present invention, which pertains to the composition using both 2-o-α-glucopyranosyl-L-ascorbic acid and glucosidase, thus it has very poor whitening and wrinkles reduction functions.

A better understanding of the present invention may be obtained through the following Examples, Comparative examples, and Preparation Examples which are set forth to illustrate, but are not to be construed as the limit of the present invention. Furthermore, it is to be understood that various modifications of Examples and Preparation Examples will be apparent to those skilled in the art without departing from the spirit of the invention.

### EXAMPLE 1

### Evaluation of Stability of an External Composition for Skin According to the Present Invention

Stabilities of 2-o-α-glucopyranosyl-L-ascorbic acid and ascorbic acid in an aqueous solution were compared.

\textcircled{1} 2-o-α-glucopyranosyl-L-ascorbic acid, \textcircled{2} ascorbic acid, and \textcircled{3} 2-o-α-glucopyranosyl-L-ascorbic acid and 10 units of α-glucosidase were added to 50 mM potassium phosphate buffer solution (pH 7.0) so that a concentration of each component was 1 mM, to produce test groups. The test groups were stored at 37° C., sampled every day for 5 days, and quantitatively analyzed.

The quantitative analysis (HPLC analysis) for test materials was conducted under conditions described in the following Table 1. An amount of remaining 2-o-α-glucopyranosyl-L-ascorbic acid or ascorbic acid which was not degraded was reduced by an initial concentration, and stability was evaluated using the ratio.

TABLE 1

| Column | Inertsil ODS-3 column (GL sciences Inc, Tokyo) |
|---|---|
| Mobile phase solvent | 0.1 M potassium phosphate-phosphoric |
| | acid buffer solution (pH 2.0) |
| Moving speed | 0.7 mL/min |
| Detection condition | UV absorption wavelength of 240 nm |

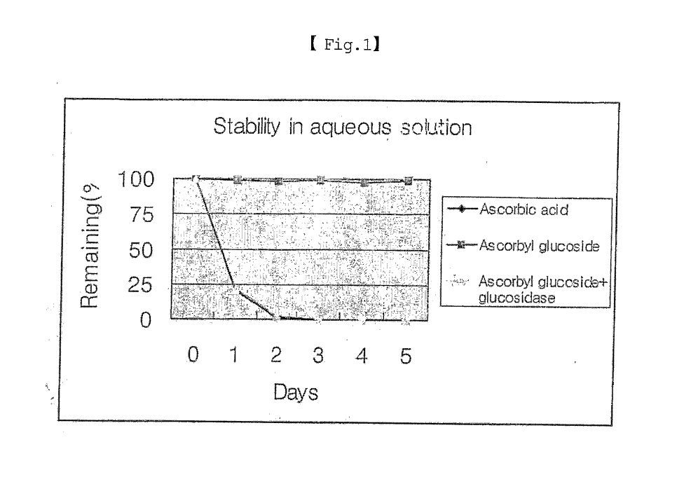

The analysis results are shown in FIG. 1. As shown in the drawing, 2-o-α-glucopyranosyl-L-ascorbic acid was seldom degraded but stably stored for 5 days. However, almost all vitamin C was degraded within 2 days. Additionally, in the case where an enzyme and 2-o-α-glucopyranosyl-L-ascorbic acid coexisted, 2-o-α-glucopyranosyl-L-ascorbic acid was degraded at a rate that was almost the same as in the test group using only vitamin C, and was seldom detected after 2 days. In this case, 2-o-α-glucopyranosyl-L-ascorbic acid was not observed even after 1 day, thus it can be seen that 2-o-α-glucopyranosyl-L-ascorbic acid was completely converted by the enzyme into vitamin C within one day. As well, it was confirmed that converted vitamin C was immediately degraded.

### EXAMPLE 2

### Test for Primary Skin Irritation Caused by an External Composition for Skin According to the Present Invention

Thirty healthy adults of both sexes were selected, and underwent a closure patch test in order to evaluate irritation of a mixed solution of 2-o-α-glucopyranosyl-L-ascorbic acid and glucosidase to the skin.

5% vitamin C solution, 10% 2-o-α-glucopyranosyl-L-ascorbic acid solution, and a mixed solution of 200 units of α-glucosidase (Sigma Inc.) and 10% 2-o-α-glucopyranosyl-L-ascorbic acid were applied in a patch form to forearms of subjects for 24 hours, and then removed. After the removal, the skin was observed for 0.5-48 hours, and the irritation was evaluated according to the standard described in Table 2a.

From Table 2a, it can be seen that the solution containing only vitamin C causes strong irritation, but the solution containing only 2-o-α-glucopyranosyl-L-ascorbic acid and the mixed solution containing 2-o-α-glucopyranosyl-L-ascorbic acid and glucosidase do not bring about primary irritation to the skin.

TABLE 2a

| Symbol | Evaluation standard |
|---|---|
| ?+ | Doubtful reaction, slight erythema etc. |
| + | Weak reaction (accompanied with no vesicles), erythema, and |
| | papula |
| ++ | Strong reaction (accompanied with vesicles), erythema, papula, |
| | and vesicles |
| +++ | Very strong positive reaction, and bullas |
| −−− | Negative |

TABLE 2b

| | Reaction | |
|---|---|---|
| | 24 | 48 |
| Test group | hours | hours |
| Buffer solution | − | − |
| 5% Vitamin C solution | ++ | ++ |
| 10% 2-o-α-glucopyranosyl-L-ascorbic acid solution | − | − |
| 10% 2-o-α-glucopyranosyl-L-ascorbic acid solution + | − | − |
| 200 units of α-glucosidase | | |

### EXAMPLE 3

### Skin Whitening Function of an External Composition for Skin According to the Present Invention

A skin whitening function of a mixed formulation of 2-o-α-glucopyranosyl-L-ascorbic acid and glucosidase according to the present invention was evaluated.

As described below, a first preparation including 2-o-α-glucopyranosyl-L-ascorbic acid and a second preparation including glucosidase were separately produced, and then contained in a vessel which had two separate spaces divided by an internal diaphragm so that contents were discharged through two separate nozzles thereof (SH Plastic, Co. of Korea). They were mixed on palms immediately before they were applied, and their whitening abilities were compared to those of comparative preparations which separately included vitamin C and 2-o-α-glucopyranosyl-L-ascorbic acid.

In detail, patches through which four circular holes having a diameter of 1.5 cm were formed were attached to each forearm of ten healthy adults of both sexes, and ultraviolet rays (Philips TL20W/12UV and TMO2/09UV lamps) having intensity of 1.5 MED were radiated from a distance of 10 cm thereonto twice per day for 2 days to form pigment in the skin. A mixed preparation in which the first preparation and the second preparation shown in Table 3a were mixed at a ratio of 1:1, and products of Comparative examples 1 to 3 were applied as test and control groups, respectively, to the pigment-deposited portion of the skin twice per day for 6 weeks, and improvement of the pigment-deposited portion was observed with the naked eye.

As shown in Table 3b, the test groups using the composition of the present invention had a whitening function that was superior to those of the control groups employing the preparations of Comparative examples, which contained only vitamin C or only 2-o-α-glucopyranosyl-L-ascorbic acid.

The formulation containing only vitamin C has a poor whitening function due to instability of vitamin C and irritation of vitamin C to the skin, and the formulation containing only 2-o-α-glucopyranosyl-L-ascorbic acid limitedly permeates through the skin due to, probably, a relatively larger molecular weight and high hydrophilicity, even though it has no ill effect on the skin. Therefore, it is believed that they have a poorer whitening function than the composition of the present invention.

TABLE 3a

| Component | First | Second | Comparative | Comparative | Comparative |
|---|---|---|---|---|---|
| (unit: wt %) | preparation | preparation | example 1 | example 2 | example 3 |
| 2-o-α- | 10.0 | — | — | 10.0 | — |
| glucopyranosyl- | | | | | |
| L-ascorbic acid | | | | | |
| Glucosidase | — | 1000 units | — | — | — |
| Vitamin C | — | — | — | — | 5.0 |
| Glycerine | 10.0 | 10.0 | 10.0 | 10.0 | 10.0 |
| Propylene | 5.0 | 5.0 | 5.0 | 5.0 | 5.0 |
| glycol | | | | | |
| Cellulose gum | 0.3 | 0.3 | 0.3 | 0.3 | 0.3 |
| Hyaluronic acid | 10.0 | 10.0 | 10.0 | 10.0 | 10.0 |
| extract | | | | | |
| pH controlling | Predetermined | Predetermined | Predetermined | Predetermined | Predetermined |
| agent | amount | amount | amount | amount | amount |
| Purified water | to 100 | to 100 | to 100 | to 100 | to 100 |

TABLE 3b

| | Improvement in pigment-deposited skin | | | |
|---|---|---|---|---|
| Test | Significantly | Slightly | No | Ill effect on |
| products | improved | improved | change | skin |
| Test | 6 | 3 | 1 | − |
| group | | | | |
| Control | − | 1 | 9 | − |
| group 1 | | | | |
| Control | − | 5 | 5 | − |
| group 2 | | | | |
| Control | 3 | 2 | 1 | 2 |
| group 3 | | | | |

### EXAMPLE 4

### Skin Wrinkles Reduction Function of an External Composition for Skin According to the Present Invention

A skin wrinkles reduction function of an external composition for skin according to the present invention was evaluated.

The products of Example 3 were used as test and control groups. That is to say, forty women having an age of 30 or higher were randomly divided into four groups (a test group, and control groups 1-3) of ten persons, and the products of Example 3 were applied to wrinkles around their eyes for 12 weeks (twice/day). Subjective evaluations by subjects were gathered using a six-grade scheme as shown in Table 4a, and the skin wrinkles reduction function was evaluated. The results are described in the following Table 4b.

TABLE 4a
Evaluation standard of skin wrinkles reduction

| | | | |
|---|---|---|---|
| Significantly deteriorated | −3 | Slightly improved | 1 |
| Deteriorated | −2 | Improved | 2 |
| Slightly deteriorated | −1 | Significantly improved | 3 |
| No change | 0 | | |

TABLE 4b

| | | Control | Control | Control |
|---|---|---|---|---|
| | Test group | group 1 | group 2 | group 3 |
| Subject 1 | 3 | 2 | 2 | 1 |
| Subject 2 | 2 | 0 | 1 | 2 |
| Subject 3 | 2 | 1 | 1 | 1 |
| Subject 4 | 3 | 1 | 0 | 1 |
| Subject 5 | 2 | 2 | 2 | 2 |
| Subject 6 | 3 | 0 | 1 | 2 |
| Subject 7 | 3 | −1 | 2 | 0 |
| Subject 8 | 2 | 2 | 2 | 0 |
| Subject 9 | 2 | 0 | 0 | 1 |
| Subject 10 | 3 | 1 | 1 | 1 |
| Average | 2.50 | 0.80 | 1.20 | 1.10 |

As shown in the above Table, after 12 weeks, in the test group using the external composition for the skin according to the present invention, the average value was 2.5 which meant that the wrinkles were improved or significantly improved. On the other hand, in the control group 2 using only 2-o-α-glucopyranosyl-L-ascorbic acid or the control group 3 using only vitamin C, the wrinkles were slightly improved. In the control group 1 which did not employ 2-o-α-glucopyranosyl-L-ascorbic acid and vitamin C, slight improvement was achieved. This is considered to result from action of other components and the effects of massage.

Accordingly, it can be seen that the external composition for the skin according to the present invention realizes wrinkles reduction by 200% or more in comparison with a conventional composition containing only 2-o-α-glucopyranosyl-L-ascorbic acid or only vitamin C.

### PREPARATION EXAMPLE

Production of various basic cosmetics using an external composition for skin according to the present invention, which separately included 2-o-α-glucopyranosyl-L-ascorbic acid and glucosidase, were described below.

Contents may be varied, but, in the following Preparation examples, a first preparation and a second preparation were used in a volume ratio of 1:1. As described above, in the composition of the present invention, 2-o-α-glucopyranosyl-L-ascorbic acid and glucosidase must be spatially separated from each other. To achieve this, different vessels may be used, or one vessel, an internal space of which is divided by a diaphragm and from which the first and second preparations are discharged in the same amount, may be used.

### PREPARATION EXAMPLE 1

### Production of an Essence Using a Composition of the Present Invention

TABLE 5

| First preparation | | Second preparation | |
|---|---|---|---|
| Component | Content (wt %) | Component | Content (wt %) |
| 2-o-α- | 10.0 | Glucosidase | 1000 units |
| glucopyranosyl-L- | | | |
| ascorbic acid | | | |
| Glycerine | 10.0 | Glycerine | 10.0 |
| Propylene glycol | 5.0 | Propylene glycol | 5.0 |
| Cellulose gum | 0.3 | Cellulose gum | 0.3 |
| Hyaluronic acid | 10.0 | Hyaluronic acid | 10.0 |
| extract | | extract | |
| pH controlling | Predetermined | pH controlling | Predetermined |
| agent | amount | agent | amount |
| Perfume | Small amount | Perfume | Small amount |
| Antiseptic | Small amount | Antiseptic | Small amount |
| Pigment | Small amount | Pigment | Small amount |
| Purified water | to 100 | Purified water | to 100 |

### PREPARATION EXAMPLE 2

### Production of a Nourishing Lotion Using a Composition of the Present Invention

TABLE 6

| First preparation | | Second preparation | |
|---|---|---|---|
| Component | Content (wt %) | Component | Content (wt %) |
| 2-o-α- | 10.0 | Glucosidase | 1000 units |
| glucopyranosyl-L- | | | |
| ascorbic acid | | | |
| Beeswax | 3.0 | Beeswax | 3.0 |
| Liquid paraffin | 4.0 | Liquid paraffin | 4.0 |
| Glycerine | 10.0 | Glycerine | 10.0 |
| Carboxy vinyl | 0.1 | Carboxy vinyl | 0.1 |
| polymer | | polymer | |
| Polysorbate 60 | 1.1 | Polysorbate 60 | 1.1 |
| Propylene glycol | 5.0 | Propylene glycol | 5.0 |
| pH controlling | Predetermined | pH controlling | Predetermined |
| agent | amount | agent | amount |
| Perfume, antiseptic, | Small amount | Perfume, | Small amount |
| pigment | | antiseptic, | |
| | | pigment | |
| Purified water | to 100 | Purified water | to 100 |

### PREPARATION EXAMPLE 3

### Production of a Pack Using a Composition of the Present Invention

TABLE 7

| First preparation | | Second preparation | |
|---|---|---|---|
| Component | Content (wt %) | Component | Content (wt %) |
| 2-o-α- | 3.0 | Glucosidase | 1000 units |
| glucopyranosyl-L- | | | |
| ascorbic acid | | | |
| Polyvinyl alcohol | 14.0 | Polyvinyl alcohol | 14.0 |
| Glycerine | 10.0 | Glycerine | 10.0 |
| Carboxy vinyl | 0.1 | Carboxy vinyl | 0.1 |
| polymer | | polymer | |
| Cellulose gum | 0.3 | Cellulose gum | 0.3 |
| PEG 4000 | 1.0 | PEG 4000 | 1.0 |
| Propylene glycol | 5.0 | Propylene glycol | 5.0 |
| pH controlling | Predetermined | pH controlling | Predetermined |
| agent | amount | agent | amount |
| Perfume, antiseptic, | Small amount | Perfume, | Small amount |
| pigment | | antiseptic, | |
| | | pigment | |
| Purified water | to 100 | Purified water | to 100 |

### PREPARATION EXAMPLE 4

### Production of a Nourishing Cream Using a Composition of the Present Invention

TABLE 8

| First preparation | | Second preparation | |
|---|---|---|---|
| Component | Content (wt %) | Component | Content (wt %) |
| 2-o-α- | 10.0 | Glucosidase | 1000 units |
| glucopyranosyl-L | | | |
| ascorbic acid | | | |
| Beeswax | 7.0 | Beeswax | 7.0 |
| Liquid paraffin | 10.0 | Liquid paraffin | 10.0 |
| Glycerine | 10.0 | Glycerine | 10.0 |
| Carboxy vinyl | 0.1 | Carboxy vinyl | 0.1 |
| polymer | | polymer | |
| Polysorbate 60 | 1.3 | Polysorbate 60 | 1.3 |
| Propylene glycol | 5.0 | Propylene glycol | 5.0 |
| pH controlling | Predetermined | pH controlling | Predetermined |
| agent | amount | agent | amount |
| Perfume, antiseptic, | small amount | Perfume, | small amount |
| pigment | | antiseptic, | |
| | | pigment | |
| Purified water | to 100 | Purified water | to 100 |

### INDUSTRIAL APPLICABILITY

An external composition for skin according to the present invention includes 2-o-α-glucopyranosyl-L-ascorbic acid, and glucosidase decomposing it to generate vitamin C. Therefore, the composition avoids problems related to instability and irritation of the skin of a conventional formulation which contains only vitamin C, and can be used as the external preparation for the skin, which has intrinsic desirable functions of vitamin C, such as high skin whitening and wrinkles reduction functions.

1. An external composition for skin, comprising 2-o-α-glucopyranosyl-L-ascorbic acid and α-glucosidase spatially separated from each other as effective components.

2. The external composition as set forth in claim 1, wherein the content of 2-o-α-glucopyranosyl-L-ascorbic acid is 0.05-20.0 wt %, and the content of α-glucosidase is 25-50 units per 1 wt % of the 2-o-α-glucopyranosyl-L-ascorbic acid.

3. The external composition as set forth in claim 1 or 2, wherein the 2-o-α-glucopyranosyl-L-ascorbic acid is obtained from nature sources or is biochemically synthesized.

4. The external composition as set forth in claim 1 or 2, wherein the α-glucosidase is added in the form of enzyme purified from a plant or a microorganism, or in the form of coarse extract thereof.

5. The external composition as set forth in claim 4, wherein the plant is one or more selected from a group consisting of rice, barley, oat, and almond.

6. The external composition as set forth in claim 4, wherein the microorganism is one or more selected from a group consisting of Aspergillus, Saccharomyces, and Zymomonas.

7. The external composition as set forth in claim 1 or 2, wherein the 2-o-α-glucopyranosyl-L-ascorbic acid and the α-glucosidase are spatially separated from each other in one vessel, or contained in two different vessels, respectively.

8. A cosmetic for skin whitening, comprising the external composition according to claim 1 or 2.

9. A cosmetic for wrinkles reduction, comprising the external composition according to claim 1 or 2.
